(19) United States
(12) Patent Application Publication
(10) Pub. No.: US 2009/0155894 A1
(43) Pub. Date: Jun. 18, 2009

(54) Electrokinetic Thermal Cycler and Reactor

(76) Inventors: Steven A. Soper, Baton Rouge, LA (US); Dimitris E. Nikitopoulos, Baton Rouge, LA (US); Michael C. Murphy, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/090,396

(22) PCT Filed: Oct. 17, 2006
(86) PCT No.: PCT/US06/40428

§ 371 Date: Apr. 16, 2008

Related U.S. Application Data
(60) Provisional application No. 60/727,697, filed on Oct. 17, 2005.

(51) Int. Cl. C12M 1/02; B01J 19/00; G01N 21/00

(52) U.S. Cl. 435/293.1; 422/198; 422/68.1; 422/55

(57) ABSTRACT

Microfluidic devices are disclosed for carrying out cyclic or iterated reactions such as PCR, LDR, and other cyclic or iterated reactions. A microchannel forms a closed loop, through which a reaction mixture may be thermally cycled an arbitrary number of times. Flow is preferably mediated primarily by electrokinetics. Multiple temperature zones may be employed along the course of a single microchannel loop, for example for PCR. Embodiments may be compact, automated, fast, and operable in continuous-flow mode. Real-time reaction monitoring may optionally be used.

(In countries other than the United States:) The benefit of the 17 Oct. 2005 filing date of U.S. patent application 60/727,697 is claimed under applicable treaties and conventions. (In the United States:) The benefit of the 17 Oct. 2005 filing date of provisional patent application 60/727,697 is claimed under 35 U.S.C. § 119(e).

The development of this invention was partially funded by the United States Government under grants R01-EB002115 and R01-HG01499 awarded by the National Institutes of Health, and grant EPS-0346411 awarded by the National Science Foundation. The United States Government has certain rights in this invention.

### TECHNICAL FIELD

This invention pertains to thermal cyclers and reactors, particularly to microscale thermal cyclers and reactors, and to other microscale cyclers.

### BACKGROUND ART

A major scientific advance useful in biological, biochemical, bioterrorism defense, and forensic applications has been the application of microfluidic devices to the polymerase chain reaction (PCR), other cyclic or iterated reactions, and other cyclic or iterated reaction systems. Such reactions require stringent control of process parameters such as temperature, reagent concentrations, buffers, and salts to run efficiently. In particular, PCR reactions should be carried out at precisely controlled temperatures. PCR is typically based on three (or fewer) discrete, multiply-repeated steps: denaturation of a DNA template, typically at approximately 95° C.; annealing of a primer to the denatured DNA template, typically at approximately 55° C.; and extension of the primer with a polymerase, typically at approximately 72° C.; to create the desired nucleic acid complementary to the template. More generally, typical temperature ranges for three-cycle PCR are about 90-95° C., about 55-65° C., and about 70-75° C. Typical temperature ranges for two-cycle PCR are about 90-95° C., and about 55-75° C. Typical temperature ranges for two-cycle LDR are about 90-95° C., and about 60-65° C.

Under ideal conditions the amount of PCR product grows exponentially with cycle number. A single-molecule DNA sample undergoing 20 PCR cycles could produce in excess of one million DNA copies. While this theoretical limit is never attained, it is nevertheless the case that, as a practical matter, about 20 or so cycles will produce an adequate amount of amplified DNA product for many purposes.

Most existing commercial PCR devices employ a “batch” approach to thermal cycling, in which wells in a single thermal block are loaded with sample and reagents, and the entire thermal block is successively cycled through the desired temperature sequence. The thermal inertia of the specimen and the heating block lead to much time being spent in transition between the desired reaction temperatures.

Reducing the thermal block and specimen size would reduce thermal inertia, thereby speeding up the heating and cooling to the desired temperatures for PCR cycles. A conventional PCR amplification requires about 2 hours to complete. There is an unfilled need for faster PCR thermal cycling techniques, for uses such as the timely and accurate identification of a suspected bioterrorism agent, and the rapid identification of pathogens in clinical and public health settings.

U.S. Pat. No. 6,514,750 discloses the miniaturization of a thermal block used in batch mode. Fluorescence may be used to monitor PCR progress in real-time.

To monitor PCR amplification optically, a synthetic primer may be introduced that includes both fluorescent and quenching groups close to one another. The quenching group suppresses fluorescence from the fluorescent group. But during the PCR process, the exonuclease activity separates the primer's fluorescent and quenching moieties, allowing fluorescence when the sample is optically probed. In this way the amount of primer may be monitored. See, e.g., U.S. Pat. No. 6,174,670.

Fluorescence monitoring requires excitation and fluorescence detection hardware and associated electronics, in addition to the thermal management equipment. Even when the thermal block is miniaturized, these components tend to make conventional “batch” PCR amplification devices non-portable.

Chemiluminescent monitoring of PCR reactions has also been reported. See, e.g., U.S. Pat. No. 6,346,384. Although chemiluminescence does not require optical excitation equipment, it still requires sensitive optical detection hardware.

Electrical conductivity probes have also been used for monitoring PCR progress, as for example in U.S. Pat. No. 6,638,716.

Monolithic microfluidic devices have been reported that use electrokinesis, particularly electroosmosis, to transport specimens between microchannels and reservoirs, for example in PCR. See, e.g., U.S. Pat. No. 6,670,153. Microfluidic systems are generally well-suited for PCR because they can permit rapid temperature changes. Because microfluidic elements such as microchannels and reservoirs are small compared to the mass of the substrate in which they are fabricated, heat may be highly localized, e.g., heat dissipates into and from the substrate before it affects other fluidic elements within the device. See also U.S. Pat. Nos. 6,261,431, 6,387,234, 6,905,583, 6,399,389, 6,180,372, and 6,428,987, and published United States Patent Application 20010041357.

U.S. Pat. No. 6,875,619 discloses lining portions of a microchip's microchannel with specific binding ligands to capture target analytes.

U.S. Pat. No. 6,413,766 discloses a microchip thermal cycling device in which the sample is heated or cooled by rapidly heating or cooling the entire chip, or large portions of the chip. Temperature could be varied by “non-contact” means: heating by infrared radiation lamps and cooling with chilled compressed air. In addition to using pressure gradients and electroosmotic flow, the expansion and contraction of liquid within the microchannel during thermal cycling could also be used to transport the sample through microchannels.

Most of these prior devices are fundamentally linear, with an initial reservoir or reservoirs connected to a long microchannel. The long microchannel may be straight, curved, serpentine, or spiral, and may optionally have branches connected to secondary reservoirs. But the microchannels in prior devices have been essentially linear, in the sense that a primary channel leads from an initial reservoir, along a defined path, and ultimately to a terminus to which the PCR reaction products are directed. A limitation of such a design is that the number of cycles is pre-determined by the chip's microchannel architecture, and may not be varied up or down to adapt to the characteristics of particular samples.

U.S. Pat. No. 6,586,233 discloses a “closed-loop” device that heats and cools a liquid specimen by transporting it through two thermal zones by thermal convection, in a “convective siphon.” By its nature, this device relies upon convection to drive fluid flow.

U.S. Pat. No. 5,296,114 discloses the use of synchronized, cyclic electrokinetic flow to conduct separations; see also Eijkel, J. et al., “Cyclic Electrophoretic and Chromatographic Separation Methods,” Electrophoresis, vol. 25, pp. 243-252 (2004); and Choi, J. et al., “Electrophoretron: a New Method for Enhancing Resolution in Electrokinetic Separations,” J. Chromatogr. A, 2001, v. 924, pp. 53-58.

B. Giordano et al., Analytical Biochemistry, vol. 291, pp. 124-132 (2001) reported a PCR cycle time of about 15 seconds per cycle. M. Hashimoto et al., “Rapid PCR in a Continuous Flow Device,” Lab Chip., vol. 4, pp. 638-645 (2004) reported a PCR cycle time of about 5.2 seconds per cycle for a 500 bp comparison target product. Hashimoto et al. employed a pressure-driven system, which is subject to loss of sample due to dispersion induced by laminar flow, and requires higher operating pressures, imposing more stringent requirements of structural integrity. There is an unfilled need for the ability to conduct faster PCR reactions, in a system that does not have such disadvantages.

### DISCLOSURE OF INVENTION

We have discovered microfluidic devices useful for carrying out cyclic or iterated reactions such as the polymerase chain reaction (PCR), and other cyclic or iterated reactions or processes. A microchannel forms a closed loop, rather than a linear path, through which a specimen or reaction mixture may be thermally cycled an arbitrary number of times, by continuing its passage through the same closed loop multiple times. Flow is preferably mediated primarily by electroosmosis. Multiple temperature zones may be employed along the course of a single microchannel loop. For example, when the invention is used for PCR there will typically be three different temperature zones along the course of the loop, one for denaturing, one for annealing, and one for chain extension. The number of cycles is not pre-determined by the architecture of the chip, but may be adjusted up or down as needed for a particular application, reaction, or sample. Cycling reactions other than cycling through different thermal zones may also be employed, if desired.

Embodiments of the invention may be compact, automated, fast, and operable in continuous-flow mode. The thermal cycling micro-reactor is capable of driving reaction mixtures rapidly to different temperatures, and optionally permits integrated, real-time reaction monitoring. Reaction monitoring may be used, for example, to efficiently terminate thermal cycling once adequate product is obtained, or to extend cycling when insufficient product has been made, or to add additional reagent(s) as needed.

A major advantage to using electrokinetically-driven flow (i.e., flow driven by electroosmosis, electrophoresis, or both), rather than pressure-driven flow, is that properly controlled electrokinetics leads to plug flow, while pressure-driven flow is typically laminar in microfluidic devices. Electrokinetic plug flow produces more uniform results. By contrast, fully-developed, pressure-driven, laminar flow has a parabolic velocity profile, leading to significant amounts of reaction product spreading out along the length of the channel, and hence less uniform results.

The novel system allows a reaction mixture to be held for an arbitrary duration at any temperature along the cycle; to travel through an arbitrary number of discrete temperature zones along the path of the cycle; to control the number of cycles; and to add reactants easily to the reaction mixture when needed during the cycling process.

We have successfully built a working prototype embodiment of the invention. The prototype comprised two basic components. The first component was a base unit incorporating a power supply, thermal management elements, detection elements, control circuits, and a computer interface. The second component was a micro-fluidic/micro-electronic reactor chip that was inserted and clamped into a receptacle in the base unit.

The prototype embodiment has successfully conducted “real-time” polymerase chain reaction (RT-PCR) amplifications faster than any prior device of which the inventors are aware. For example, cycles may be conducted in under 15 seconds, under 10 seconds, or under 5 seconds. A prototype has successfully conducted PCR amplifications with a cycle time of about 5 seconds. The PCR amplifications were highly accurate, with a low error rate. Conventional PCR thermal cyclers are not well-adapted for “point-of-use” operation. Instead, specimens to be amplified must generally be transported to a laboratory facility for analysis. The present invention allows rapid, point-of-use operation, which has substantial benefits for applications such as bioterrorism defense and crime scene investigations (e.g. DNA forensics).

The invention offers advantages in conventional laboratory settings as well. Its speed in amplifying DNA can enable rapid, unambiguous identification of pathogens, which can have significant clinical and public health consequences. Shortcomings of existing equipment include limited versatility and high hardware and reagent costs. The microscopic scale of this invention vastly reduces requirements for expensive reagents. Embodiments may be mass-produced cheaply by simple embossing techniques using polymeric substrates, or by injection molding.

The prototype embodiment successfully carried out PCR amplifications in continuous flow mode, using synchronized electrokinetic pumping. Problems associated with hydrodynamic flow in a continuous flow PCR format were avoided, such as leakage from the high back pressure required for hydrodynamic pumping in a long microchannel with a small cross-sectional area, sample dilution due to the hydrodynamic flow profile, and the need for off-chip pumps. Using an electrokinetic, synchronized format for continuous flow PCR also allowed adjusting the number of PCR cycles without redesigning the microchip. Compared to block-type PCR thermal cyclers, the prototype chip reduced PCR sample volume from 10-μL to ˜0.5 μL. Further reductions in sample volume are possible. More generally, the volume of the closed loop reactor is preferably between about 50 μL and about 10 mL, more preferably between about 1 mL and about 10 μL. The chip may, if desired, be easily integrated with microchip electrophoresis without active mechanical valving, simplifying operation of the integrated device. The short effective channel length that may be used (1.9 cm in the prototype) allows small power supplies to be used to generate the electric field, thereby reducing the footprint of the device, which has particular advantages for field-deployable applications. The speed of the PCR amplification reaction may readily be adjusted by altering the electric field strength and controlling the direction and magnitude of the electroosmotic flow.

### BRIEF DESCRIPTION OF THE DRAWINGS

FIG. 1 depicts schematically a prototype embodiment of the invention.

FIGS. 2A through 2D depict schematically a synchronized, cyclic, continuous-flow PCR reaction in the prototype device

FIG. 3 depicts schematically an electrophoretron embodiment of the invention.

### MODES FOR CARRYING OUT THE INVENTION

We have used the prototype to conduct “real-time” PCR, including “real-time” monitoring of the reaction, through a three-temperature reaction cycle of denaturing at 95° C., annealing at 55° C., and extension at 72° C.

In addition to PCR, other cyclic processes may be implemented with this invention, for example, for supporting cell lysis, cell incubation, or ligation detection reaction (LDR), as well as combinations of modules to conduct these reactions, either in single or multiplexed configurations (e.g., multiple identical or different micro-fluidic elements on a single micro-chip module).

The micro-reactor chip may incorporate one or more of several features:

Electrokinetically-driven, continuous, cyclic flow of reactant mixture(s) in a closed-loop (“racetrack”) micro-channel geometry. This may be achieved in different ways, including for example the following approaches:

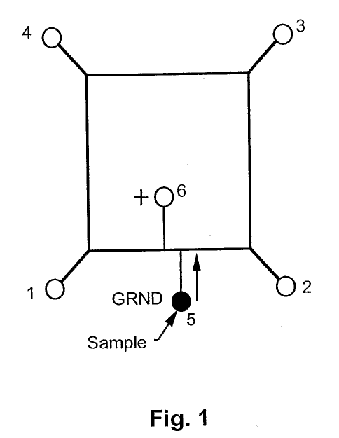

Alternating voltages at a plurality of electrodes around the “racetrack,” e.g., placing an electrode at each of the 4 corners of a “square racetrack,” as illustrated in FIG. 1, and imposing time-varying, synchronized potential differences across the electrode pairs after loading the sample; or

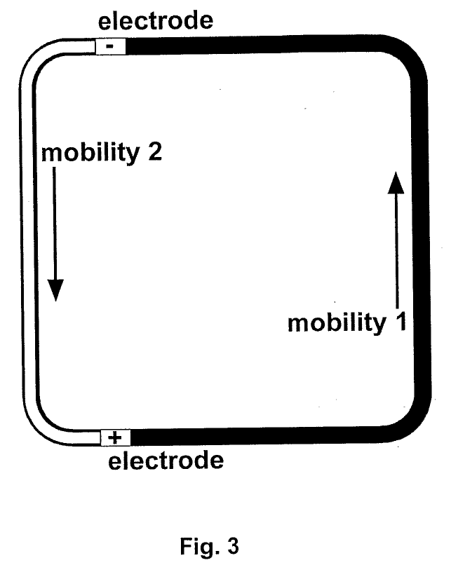

Imposing a steady potential difference on two electrodes that are positioned so as to separate a closed loop channel into two segments with different, but complementary mobilities and channel lengths, for example using an electrophoretron similar to that otherwise described in Choi, J. et al., “Electrophoretron: a New Method for Enhancing Resolution in Electrokinetic Separations,” J. Chromatogr. A, 2001, v. 924, pp. 53-58. This approach is illustrated in FIG. 3.

The segment-by-segment approach of the first alternative above reduces the distance over which an electric potential must be applied to induce electrokinetic flow. Thus the device can operate at lower voltages than would a non-cyclic counterpart. The ability thus to use a smaller power supply enhances the device's portability. The novel device does not require a pressure gradient, which enhances mechanical stability and robustness. The high pressure gradients required by some microfluidic devices can lead to loss of structural integrity.

Reaction mixtures can be subjected to temperature cycling that is far more rapid than may be achieved with conventional equipment. The reaction mixture moves, preferably continuously, between different steady-state temperature zones, zones that preferably co-exist simultaneously on different parts of the chip. This method of temperature control is far faster than is found in conventional PCR thermal cycler devices.

The invention also facilitates improved real-time monitoring of reaction products via optional detector(s) integrated into the microchannel racetrack. Such detection may optionally operate with or without fluorescence equipment, and with or without labeled reaction products.

The invention provides the flexibility to vary the number of reaction cycles, a flexibility that is absent from existing pressure- or electrokinetically-driven devices that have fixed channel lengths, without a closed-flow loop. The invention also affords the option of adjusting the reaction mixture by electrokinetically mixing the sample and additional reagents in the closed-loop micro-channel “racetrack” through appropriate voltage-control actions.

Manufacture of microfluidic reactor chips through micromanufacturing techniques otherwise known in the art, such as hot-embossing or injection-molding, using polymeric materials such as polycarbonate (PC) or poly(methyl methacrylate) (PMMA) or co-polymers (e.g. COC). The inexpensive mass-production of many chips from a single mold master makes the chips potentially disposable, and can help reduce the potential for sample contamination. The polymer used may be chosen based on what is optimal for a specific application; polymers with different electroosmotic properties may be selected for different applications.

Control of the combined electroosmotic and electrophoretic motion of samples in a micro-fluidic reactor chip by, for example, chemical or irradiative (e.g., UV) preconditioning of the polymer surface, to suit the needs of a specific application. The benefits include: (a) the flexibility of using otherwise similar, mass-produced chips formed from different polymers, co-polymers, or mixtures, to accommodate the electrophoretic properties of different biological samples; and (b) the ability to induce electrokinetically-driven, cyclic flow in a closed loop channel formed from a single material with only two electrodes, by preconditioning one branch of the channel, as discussed further below and as illustrated in FIG. 3.

In-situ, conductivity-based, real-time detection of reaction products (e.g. PCR-amplified DNA oligomers) directly on the micro-fluidic reactor chip itself. For example, integrated electrode pairs may be used for conductivity-based detection of amplified DNA products with a low-conductivity, bio-compatible buffer. The benefits include: (a) the ability to continuously monitor and identify reaction products; (b) the ability to reduce total reaction time by terminating the reaction after the desired level of product has been produced, as determined by continuous or periodic monitoring of products; (c) simple, compact, and inexpensive detection as compared to more traditional fluorescence detection, which typically requires a combination of laser, optics, and optical detector; (d) no need to label reaction products, thus lowering the cost and complexity of bioassays. To minimize interference with electrokinetic flow, the detection field is preferably orthogonal to the electrokinetic driving field, has a low potential drop, and is AC rather than DC.

Conducting thin metal films may be electrodeposited onto the polymer substrate to form electrodes and electrically conducting paths.

The electrokinetic properties of some reaction components and products may change as a reaction proceeds, while others may not. Real-time monitoring of the reaction products allows the applied voltage to be modified as needed to keep the position of a reaction product synchronized with the driving field. To take a common example, the electrophoretic mobility of PCR reaction products (oligonucleotides of 20 base pairs or more) does not change substantially in free solution as the chain length increases, so typically the applied voltage need not be modified to accommodate PCR reaction products of different sizes. However, it still may be adjusted as needed to best match the observed mobility of the PCR products in actual use.

Optionally, PCR reaction products or other reaction products may be monitored by various means otherwise known in the art. A preferred method for monitoring in many circumstances is label-less conductivity detection. Integrating reaction monitoring with a closed-loop (“racetrack”) thermal cycler or other reactor allows one to optimize operation in real time, and to minimize reaction completion time. The mass detection limit of the conductivity detector used in a prototype embodiment of the racetrack thermal cycler for λ-DNA was about 100 ng/μL (copy number ˜2×10⁹/μL), with a signal-to-noise ratio of ˜3. The molar detection limit was ˜3 nmol/L. Optionally, monitoring techniques such as conductivity detection may be used to discriminate between different molecules, such as proteins and DNA.

Electrolysis in an electrokinetically driven device can generate gas at the anode, the cathode, or both. Such gas is generally undesirable, as it can present problems both for operation of the cycler, and for conductivity detection. To reduce gas accumulation in the micro-channel, reservoirs (e.g., ˜20 μL) may be used to hold gas, or the electrodes may be positioned close to the upper surface of the buffer in these reservoirs. When gas is generated, it then moves from the buffer, avoiding migration into the flow stream. In addition, the salt concentration in the running buffer may be kept low, e.g. by reducing the KCl concentration in the PCR “cocktail” to slow the gas generation rate. Electrolysis also produces OH⁻ at the cathode and H⁺ at the anode. Too great a concentration of either could overwhelm the buffer's ability to maintain a steady pH. Reducing the KCl concentration, even to zero, was observed not to substantially reduce the amount of PCR product produced by the system, while substantially reducing the current and therefore the amount of unwanted electrolysis products.

To reduce the formation of gas bubbles due to heat, especially within zones whose temperature approaches the boiling point, such as the 95° C. zone in PCR, it can be helpful to add a component that will increase the boiling point without substantially interfering with the desired reactions, e.g., 2% ethylene glycol.

### EXAMPLE 1

Microchip Design and Fabrication. A prototype embodiment of the invention has been constructed. The channel size in the PCR reactor microchip prototype was 100 μm in width, 70 μm in depth, and 7.9 cm long, for a total reactor volume of 0.55 μL. Access channels positioned at each corner of the reactor had the same dimensions as those of the reactor channel.

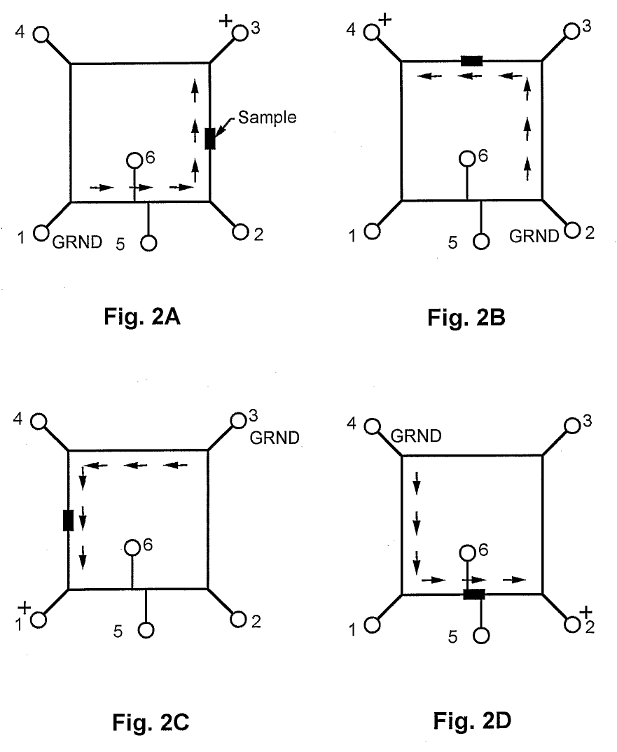

This prototype embodiment, and the synchronized, cyclic, continuous-flow PCR process are depicted schematically in FIG. 1, and FIGS. 2A through 2D. FIG. 1 depicts sample injection. Reservoirs 1-4 accommodated electrodes for applying voltages between points 1 and 3, as well as between points 2 and 4, for moving the DNA plug through the three temperature zones in a synchronized fashion. A DNA sample was injected into reservoir 5, and a voltage was applied across the electrodes in reservoirs 5 and 6. Sample was moved across the reactor channel to fill the crossed T injector. FIGS. 2A through 2D depict sample cycling. The cycle includes four phases: (A) a voltage was applied across the electrodes in reservoirs 1 and 3, moving the DNA plug from the bottom channel to right channel; (B) a voltage was applied between reservoirs 2 and 4, moving the sample from the right channel to the top channel; (C) a voltage was then applied between reservoirs 3 and 1, moving sample from the top channel to the left channel; and (D) a voltage was then applied between reservoirs 4 and 2, causing the sample to return to the starting position. The voltages for injection and cycling may be adjusted to set the linear velocity within the channels as desired. In a typical example, the injection voltage was about 800 V, and the cycling voltage was about 1500 V.

A brass mold insert was prepared by otherwise standard micromanufacturing techniques. A micro-fluidic chip was replicated from the master in polycarbonate (PC), using a commercial hot embossing system with a precision press fitted with a vacuum chamber to remove air (<0.1 bar) to enhance filling of the die. Prior to the hot embossing step, residual water in the polymer was removed by baking the PC wafers in an oven at 80° C. overnight. During embossing, the mold insert was heated to 180° C., and pressed into the PC wafer with a force of 1000 lb for 4.5 min. Then the press was opened, and the embossed polymer was removed and allowed to cool. The PC wafer was held at a constant 85° C. throughout the de-molding process. Following embossing, the devices were rinsed copiously with isopropanol to remove residual mold-release agent. The final device was assembled by thermally annealing the molded piece to a cover plate (0.5-mm thickness) made from PC, at 150° C. in a circulating air oven for 20 min.

### EXAMPLE 2

Temperature Control: Each of three temperature zones was heated by a surface-mounted resistor array. Temperatures were monitored by four K-type thermocouples embedded within each array, close to the contact surface between the heater and the chip. Zone temperatures were maintained within 0.5° C. by a PID type control loop. To maintain as uniform a temperature as possible, and to reduce heat transfer between zones, each heater was isolated by an air gap from neighboring heaters. Because some heat transfer is inevitable, the resistance of the end resistor of any zone that is adjacent to a higher temperature zone may be increased to reduce some of its heat generation. By moving the sample through independent temperature zones, the time delay due to temperature ramping is greatly reduced. The delay, therefore, depends principally on the sample migration time from zone to zone, and heat transfer to or from each zone. The surface-mounted heaters were sealed onto a separate polycarbonate block, which contained a pocket to accommodate the fluidic chip. The fluidic chip was brought into conformal contact with the heater arrays using a single Teflon screw and a nut, to allow easy replacement of the fluidic module without mounting the heaters to a new chip.

### EXAMPLES 3 AND 4

Surface Modification of PC Microchannels: Various surface modification techniques otherwise known in the art may be used; the surface modifications should be stable in the temperature range of interest, e.g., that used for typical PCR or LDR reactions.

One approach employed a dynamic coating to modify the microchannel walls to alter the magnitude and direction of electroosmotic flow (EOF). A principal goal is that the EOF should not oppose the electrophoresis of charged species, e.g., DNA. For example, EQF for unmodified polycarbonate (PC) is positive. It is desirable to reverse the direction of the EOF for DNA, because the direction of electrophoresis for DNA is negative. The voltage needed to move DNA through the channels is reduced, and dispersion of the DNA plug due to counter-propagating flows of buffer and DNA is also reduced.

Hexadimethrine bromide (Polybrene, PB) was used as a dynamic coating material. The channel was first rinsed with 0.1 M NaOH and deionized water for 4 min each. Once preconditioned, the channel was filled with 5% PB solution. After a 10-min incubation, residual PB solution was removed by vacuum. Finally, the coated device was allowed to stand for 15 min before use.

### EXAMPLE 5

Another approach is used in the electrophoretron embodiment of the invention, depicted in FIG. 3. In an electrophoretron, it is desirable to have opposite mobilities in the two branches of the channel loop, leading to uniform flow overall. Thus it is desirable to have a surface modification that will reverse electroosmotic mobility in one segment of the channel loop, while leaving that in the remaining portion unmodified. It is preferred that the modification should be one that may be implemented after closing the micro-fluidic chip, with the cover bonded to it. Amination of many polymers, including PC and PMMA (i.e., attaching amine groups to the polymer surface) reverses the direction of electroosmotic mobility at appropriate solution pH. For example, carboxylic groups may be attached to the polymer surface via UV-activation, and the carboxylic groups bonded to amine-terminations using 1-ethyl-3-(3-dimethylaminopropyl) carbodiimide hydrochloride (EDC) as a catalyst. There are other means known in the art for attaching amine groups to a surface; an advantage of the UV method is that it may be used to modify a segment of the channel after the chip has been closed. In addition to such surface coatings, other surface modifications such as modifying the geometry on the nanoscale can alter electrokinetic mobility.

A UV-mask was used to block UV radiation from the part of the channel and the corresponding part of the cover (before bonding) where modification was not desired. After irradiation, the cover was aligned with the substrate to match the corresponding transition boundaries between UV-activated and non-activated portions of the two parts. Then the two parts were bonded, e.g., by, thermal bonding. Then the amination solution was circulated through the now-closed channel to aminate the UV-activated portions. The UV-activated portions were aminated to have their electroosmotic mobility reversed, while the non-activated part remained unmodified and retained positive electroosmotic mobility.

After hot embossing, the micro-fluidic chip components were first cleaned with detergent and distilled water. Then they were sonicated for a few seconds on both sides to remove particulate matter, rinsed with propanol, and rinsed again with distilled water. The parts were dried using a pure air jet.

The substrate with the micro-channel and the cover were fitted with the UV-mask, and exposed to UV-Light (254 nm) for 30 minutes at a distance of 1 cm from the UV light source at a power density of 15 mW/cm². The conditions of the exposure were based on the magnitude of the reversed electroosmotic mobility desired. After exposure, the parts were cleaned again with distilled water, and dried.

Following alignment, the cover was thermally bonded to the substrate with the channel. The components were aligned by means otherwise known in the art, for example by lining up under a microscope scribed line marks (a few tens of microns wide and deep) marking the line dividing the UV-exposed and non-exposed portions of the pieces. These marks were placed so that they did not interfere with bonding in the working area of the chip. For mass-production alignment can be achieved through embossed complementary alignment features on the different parts.

An ethylenediamine solution was used for amination. The solution comprises 1 mL of phosphate buffer (pH=7), 45 μL of ethylenediamine solution (99% concentration, as obtained commercially), and 1 mg of EDC powder. The solution was loaded into the channel with a syringe pump and allowed to stand overnight. Upon completion of the amination step the solution was washed with distilled water.

FIG. 3 depicts schematically an electrophoretron cycling reactor. The micro-fluidic element comprises a closed meso- or micro-channel loop having arbitrary shape and cross-section. Electrodes are positioned along the closed loop channel, dividing it into two branches, generally (but not necessarily) having unequal lengths. One branch of the channel has an electroosmotic mobility that differs from that of the other branch, and is preferably opposite in sign. If the electroosmotic mobilities of the two channel branches are chosen so that the effective electrokinetic velocity (combined electroosmotic and electrophoretic velocity) of the charged species of interest (e.g. DNA) remains unidirectional throughout the closed channel loop when a potential difference is applied across the electrodes, then the charged species will travel continuously around the loop as indicated by the arrows.

It is preferred that the electrophoretron embodiment have the following characteristics: (a) unidirectional flow (e.g., uniformly clockwise or counterclockwise) along the two branches of different mobility, to promote continuous cyclic flow without reversals; and (b) Highly uniform velocity distribution across the length of the channel loop.

The positions of the electrodes, and the electric potentials applied to the electrodes, should be selected to satisfy these criteria. Optimal voltage potentials for the electrodes may readily be determined for a particular application of interest (e.g., for PCR) through routine testing. The electrodes should preferably “straddle” (or be close to) the electroosmotic mobility transition boundaries. Placing the electrodes too far from these boundaries can eliminate cyclic operation, or reduce its efficiency.

### EXAMPLES 6-10

Mathematical modeling may optionally be used to determine approximate values for electrode positioning and potentials. A simple analysis is presented below, based on one such model. This model assumes a channel of circular cross-section, disregards effects in the proximity of the electrodes, disregards surface charge discontinuities at channel branch junctions, and disregards the effect of bends in the fluid path. Under these assumptions, an analytical solution of combined electroosmotic and hydrodynamic, fully-developed flow in a straight tube is a good approximation. The analytical solution is based on the underlying assumptions for a Boltzmann distribution of ions in the diffuse layer, the Debye-Huckel linear approximation, and the assumption that double layers are thin relative to other length scales.

Generally, each channel branch, j (=1, 2) may have a different length, Lⱼ, and electroosmotic mobility μₑₒⱼ. We assign the subscript 1 to a first channel branch and the subscript 2 to a second channel branch. The velocity distribution in the j^{th} channel branch is then given as:

$${u}_{j} ( r ) = \frac{ΔΦ}{{L}_{j}} {μ}_{coj} - \frac{{a}^{2}}{4 μ} \frac{Δ p}{{L}_{j}} [ 1 - {( \frac{r}{a} )}^{2} ] \qquad ( 1 )$$

where μ is the dynamic viscosity of the fluid, ΔΦ is the potential difference applied between the electrodes and, Δp is the hydrodynamic pressure difference. For simplicity, the inner radius of the tube, a, is assumed to be the same for both branches, although it does not in general need to be the same.

To achieve bulk cyclic motion of the buffer fluid, the volumetric fluxes of the fluid in the two branches must be equal and opposite in sign, as a consequence of the conservation of mass. We obtain a relationship between the induced hydrodynamic pressure difference and the applied voltage:

$$Δ p = \frac{8 μ}{{a}^{2}} ΔΦ \frac{{\sum }_{k} \frac{{μ}_{eok}}{{L}_{k}}}{{\sum }_{k} \frac{1}{{L}_{k}}} \qquad ( 2 )$$

Thus the velocity profile of the fluid that is expected in the j^{th} branch becomes:

$${u}_{j} ( r ) = \frac{ΔΦ}{{L}_{j}} \{ {μ}_{eoj} - 2 \frac{{\sum }_{k} \frac{{μ}_{eok}}{{L}_{k}}}{{\sum }_{k} \frac{1}{{L}_{k}}} [ 1 - {( \frac{r}{a} )}^{2} ] \} \qquad ( 3 )$$

The motion in the cycler is that of a charged species (e.g. DNA) subject to electrophoresis. In an electroosmotic/hydrodynamic velocity field the species of interest, with electrophoretic mobility μₑₚ, will move with the local fluid velocity superposed on the electrophoretic velocity induced by the electrical field in the channel. This superposition is valid so long as the presence of these molecules does not affect the flow process and the electric field in the channels. This assumption is reasonable, given the typically low concentrations of such molecules. The velocities of the charged molecules may be estimated by:

$${u}_{cj} ( r ) = \frac{ΔΦ}{{L}_{j}} \{ {μ}_{ej} - 2 \frac{{\sum }_{k} \frac{{μ}_{eok}}{{L}_{k}}}{{\sum }_{k} \frac{1}{{L}_{k}}} [ 1 - {( \frac{r}{a} )}^{2} ] \} \qquad ( 4 )$$

where μₑₚ+μₑₒⱼ=μₑⱼ is the effective mobility.

The principal condition for cyclic operation is to maintain unidirectional motion of the charged species along the cyclic path at every location in the channel cross-section. Considering that the profile of (4) has an extreme value at the center of the tube (r=0), this condition is satisfied when the velocity of the species in the center of the tube and on the walls is of the same direction, and changes sign when crossing from one channel branch to the other. This will be true if the following conditions are met:

$$\begin{matrix}{μ}_{e 1} > 0 and {μ}_{e 2} < 0 \\ and & ( 5 ) \\ {μ}_{e 1} - 2 \frac{{\sum }_{k} \frac{{μ}_{eok}}{{L}_{k}}}{{\sum }_{k} \frac{1}{{L}_{k}}} > 0 and {μ}_{e 2} - 2 \frac{{\sum }_{k} {μ}_{eok}}{{\sum }_{k} \frac{1}{{L}_{k}}} < 0 & ( 6 )\end{matrix}$$

Considering these conditions, we can define three parameters on which a design may optionally be based: (1) an effective mobility ratio, MR₁₂=μₑ₁/μₑ₂, (2) an electrophoretic mobility ratio, MRₚ₂=μₑₚ/μₑ₂, and (3) a channel branch length ratio α=L₁/L₂ The effective mobilities in the channel branches should be of opposite sign. Without loss of generality, we arbitrarily choose the mobility in branch 1 to be positive, since we can always interchange the indices 1 and 2. It then follows that MR₁₂<0 and that MRₚ₂ may be positive or negative, depending on whether the electrophoretic mobility of the species has the same sign or the opposite sign as the effective mobility for branch 2. The numerical values of the ratios for which cyclic flow is expected are given by Equation (6); these values depend on the channel branch-to-length ratio. From equations (2) and (3) it follows that if

$${\sum }_{k} \frac{{μ}_{eok}}{{L}_{k}} = 0 ,$$

i.e.

$$\frac{{μ}_{co 1}}{{μ}_{co 2}} = - α ,$$

then the induced pressure difference is zero and the buffer flow should be purely electroosmotic in both branches with “top-hat” or “plug” velocity profiles, and the species velocity should be uniform over the cross-section in both branches. These conditions, along with Equation (5) give approximate optimum conditions for the modified electrophoretron, whether for use as a separation device or as a cycler. Although not preferred, the cycler should even work when the electroosmotic mobilities in the two branches are not necessarily of opposite sign.

The approximate analysis described above does not account for the effect of varying temperatures along the fluid flow path. The extent to which temperature differences in a thermal cycling reaction such as PCR (with three temperature zones) or LDR (with two temperature zones) may alter these results depends, as a practical matter, on tolerances for the required residence time in each zone. If the velocity profile for species motion is not uniform, then different portions of the cycled species may be subject to different residence times in the zones. Such variation is acceptable so long as the residence times and their relative proportions stay within tolerances for the reaction or separation in question. Specifically for PCR or LDR these tolerances are relatively broad; see for example M. Hashimoto et al., “Rapid PCR in a Continuous Flow Device,” Lab Chip., vol. 4, pp. 638-645 (2004).

Our theoretical calculations (not shown) demonstrated that there is a substantial window of acceptable parameter combinations for successfully cycling charged species.

In addition to this theoretical analysis, we have also conducted numerical, computer-based simulations of combined electroosmotic and electrophoretic flow in a realistic layout, including bends, electrodes and mobility discontinuities. The numerical simulations showed effectiveness in circulating a charged species (e.g. DNA) under both ideal and non-ideal operating conditions. “Ideal” (or preferred) conditions are those that both satisfy characteristic (a) (unidirectional flow), and substantially satisfy characteristic (b) (highly uniform velocity distribution), while those that deviate substantially from characteristic (b), although satisfying (a) are considered non-ideal (or not preferred). The four mobility combinations used in our numerical simulations are shown in Table 1.

TABLE 1

| | μₑₚ | μₑₒ_{₁} | uₑₒ_{₂} | | |
|---|---|---|---|---|---|
| Case | μm²/(V s) | μm²/(V s) | μm²/(V s) | MR₁₂ | MRₚ₂ |
| 1 | *−3.75 10⁴ | *7.1 10⁴ | *−2.56 10⁴ | −0.531 | 0.594 |
| 2 | *−3.75 10⁴ | 5.68 10⁴ | *−1.37 10⁴ | −0.377 | 0.732 |
| 3 | *−3.75 10⁴ | *7.1 10⁴ | *−1.707 10⁴ | −0.614 | 0.687 |
| 4 | *−3.75 10⁴ | *7.1 10⁴ | −5.12 10⁴ | −0.379 | 0.424 |

The four mobility combinations shown in Table 1 were intended to be realistic for a polycarbonate substrate material. Polycarbonate is an appropriate material for thermal-cycler reactor micro-fluidic chips (PCR, LDR) because of its high glass transition temperature (˜155° C.).

The values marked by asterisk in Table 1 were taken from our measurements in: J. Chen et al., “Electrokinetically Synchronized Polymerase Chain Reaction Microchip Fabricated in Polycarbonate”, Analytical Chemistry, 2005, v. 77, pp. 658-666); and N. Elmajdoub, “Surface Modification Process for EOF Reversal and EOF Measurements,” Chapter 4 of A Modified Micro-Scale Electrophoretron, M. S. Thesis (Louisiana State University, Baton Rouge, 2006) for unmodified polycarbonate, and for polycarbonate that has been surface-modified to reverse the electroosmotic mobility sign. The other values are within feasible ranges. These simulations did not account for variations in properties due to thermal effects. These simulations did not address conditions close to the expected limits of operability.

Case 1 was chosen to satisfy the theoretical condition

$$\frac{{μ}_{eo 1}}{{μ}_{eo 2}} = - α ,$$

for no hydrodynamic effects on the buffer fluid velocity profile, corresponding to the most preferred conditions according to the simple theoretical analysis. Additional detail and results can be found in N. Elmajdoub et al., “Design Aspects and Simulations of a Modified Micro-Scale Electrophoretron,” Paper IMECE2006-15365, Proceedings of ASME: 6th International Mechanical Engineering Congress and Exposition (Chicago, Ill., Nov. 5-10, 2006). Theoretical simulation results (not shown) showed a charged species that was introduced initially as a plug indeed moved around the loop as a plug. Nevertheless, unlike the case for other devices such as pressure-driven devices, or even the electrokinetic synchronous embodiment of the present invention, potential dispersion of the plug should not be a problem for the electrophoretron embodiment of this invention, because in general the sample will fill the channel and circulate through the various temperature zones multiple times regardless of possible dispersion.

Results for non-ideal cases 2 and 3, and case 4 (not shown) also demonstrated unidirectional motion for the charged species, i.e., continuous cyclic motion without reversals, even under the less-than-ideal conditions. Thus the design should be sufficiently robust to accommodate potential experimental uncertainties in manufacturing or surface modification.

### EXAMPLE 11

We have successfully demonstrated rapid Real-Time PCR(RT-PCR) in a prototype embodiment using the four-electrode configuration depicted in FIGS. 1 and 2. We loaded the device with a 10 ng/mL concentration of DNA template (48 kbp A-DNA). PCR reagents were also loaded into the device: a forward primer, which was a complement to the negative strand at position 7131-7155 (25 bp), and a reverse primer, which was a complement to the positive strand at bases 7606-7630 (25 bp) (Integrated DNA Technologies, Coralville, Iowa). The PCR cocktail also contained 10 mM of Tris-HCl (pH 8.3), 1.5 mM MgCl₂, 0.001% w/v gelatin (Sigma-Aldrich, St. Louis, Mo.), 2.5 Unit/100 μL of AmpliTaq DNA polymerase, 200 μM of each of the dNTPs (Applied Biosystems, Foster City, Calif.), and 1.0 μM of the PCR primers. No KCl was added to the PCR cocktail.

PCR products were fluorescently labeled by including the dye SYBR green in the PCR reaction cocktail. This dye does not interfere with the PCR process, but it does non-covalently interact with the generated double-stranded DNA to produce a fluorescence signature. PCR copy number was thereby monitored by observing fluorescence as a function of cycle number with an inverted fluorescence microscope.

The number of PCR “amplicons” initially increased with increasing cycle numbers, but the amplicon number reached a “plateau” after about 40 cycles. This plateau resulted from such causes as: (i) depletion of dNTPs; (ii) depletion of primers; (iii) degradation of the polymerase enzyme; (iv) end product inhibition by duplexed DNA; (v) non-specific competition for resources (production of incorrect product); or (vi) re-annealing of specific products to one another instead of to the primers (a particular problem at high product concentrations). The number of cycles until reaching plateau, about 40, does not differ substantially from that for macro-scale implementations, and depends primarily on the details of the particular PCR biochemical reagents and concentrations employed.

For the ˜500 bp amplicon, we were able to achieve a cycle time as short as 5.2 seconds per cycle over 20 cycles, for a total reaction time of only 1.7 minutes. This was comparable to the speed reported by Hashimoto et al. (2004), which used a pressure-driven system. The cycling time reported here and that of Hashimoto et al. (2004), both about 5 seconds per cycle, are the fastest reported cycle times of which the inventors are aware for a PCR comparison target of comparable length, ˜500 b.p.

Electrophoresis confirmed the presence of a single product band of the expected size, 500 b.p. with no spurious products observed within the sensitivity limits of the laser fluorescence detector.

The relative yield of PCR-amplified DNA product from the chip was about 50% of the yield from a conventional PCR machine using comparable PCR and electrophoresis conditions. We attribute the lower yield primarily to polymerase deactivation, or DNA loss from nonspecific adsorption to channel walls, as the surface-to-volume ratio is much higher in the novel chip than in a conventional block thermal cycler. A 50% loss in yield will, for many purposes, be more than compensated by the faster reaction times, the lower consumption of reagents, portability of the device, and ease of use.

### EXAMPLE 12

A preferred implementation incorporates thermal management features to enhance PCR yield. These thermal management features include: (1) mounting the heaters on thermally conductive blocks, e.g., copper metal; (2) placing grooves in the substrate, to better isolate the temperature zones from one another; and (3) decreasing the thickness of the substrate to produce better-defined temperature zones with sharper gradients at their boundaries. When these three techniques were applied to the device of Hashimoto et al. (2004), PCR yield increased by 370% versus that reported by Hashimoto et al. (2004); and total PCR yield increased to 72% of conventional PCR yield in a macroscale device. Similar thermal management techniques will yield similar increases in yield in the present invention.

Definitions. As used in the specification and Claims, unless otherwise clearly indicated by context, the following terms shall have the following meanings: A “closed loop,” a “closed cycle,” or similar terms denote a fluidic pathway that is adapted to allow a fluid to traverse the same path (of arbitrary shape), the same cycle, repeatedly, an arbitrary number of times that is not pre-determined by the system's architecture. A “racetrack” is a convenient metaphor for envisioning such a “closed loop.” As in an actual racetrack, there may be (and typically are) one or more entrances and exits, passages where fluid may be admitted into or withdrawn from the cycle. The term “closed” does not preclude the presence of such input and output points. Rather, the term “closed” is intended in contrast to a configuration such as a simple spiral, where the fluid enters, traverses the spiral one or more times (determined by the architecture), and exits. The term “closed” is also intended in contrast to a “linear” configuration such as one where the fluid circulates multiple times in the same general direction over the same general parts of a system, or in a more complex pathway, but not traversing precisely the same path repeatedly, and where, due to the architecture of the system, the fluid will circulate a pre-determined number of times before exiting the system—a number that is predetermined by the architecture of the system, rather than being selected by the user to fill a particular need. A “temperature controller” may heat or cool a selected zone, e.g., an electrically resistive heater, or a flow of cool air or liquid.

The complete disclosures of all references cited throughout the specification are hereby incorporated by reference, as is the complete disclosure of the priority provisional application, U.S. provisional patent application Ser. No. 60/727,697, filed 17 Oct. 2005. Also incorporated by reference are the complete disclosures of the following references by, or attributable in pertinent part to, the inventors: J. Chen et al., “Electrokinetically synchronized polymerase chain reaction microchip fabricated in polycarbonate,” Anal. Chem., vol. 77, pp. 658-666 (2005). In the event of an otherwise irreconcilable conflict, however, the present specification shall control.

What is claimed:

1. A reactor comprising:
(a) a closed loop reactor;
(b) one or more ports to admit reagents into said closed loop, and to withdraw reaction products from said closed loop;
(c) two or more temperature controllers adapted to control the temperature of said closed loop in two or more distinct zones, wherein the temperatures of different zones can differ substantially from one another;
(d) two or more electrodes in electrical contact with the interior of said closed loop, wherein said electrodes are adapted to cause the electrokinetic flow of reagents in solution through said closed loop;
wherein:
(e) said closed loop and said electrodes are adapted to allow the repeated circulation of reagents in solution through said closed loop an arbitrary number of times, as selected by a user, and to allow the solution to repeatedly pass through the different temperature zones an arbitrary number of times, as selected by a user.

2. A reactor as recited in claim 1, comprising at least three said electrodes, wherein said electrodes are positioned at intervals within said closed loop, in a configuration that is adapted to allow electrical potentials to be applied to said electrodes in a periodically varying manner, so that electrokinetic flow within said closed loop circulates in a periodic manner as the electrical potentials of said electrodes are varied.

3. A reactor as recited in claim 1; wherein said closed loop comprises interconnected first and second branches, said first and second branches meeting one another at first and second boundaries; wherein said second branch comprises a surface modification, or a different material, that alters the sign of electroosmosis within said second branch as compared to the sign of electroosmosis within said first branch; wherein said reactor comprises a first electrode positioned in the vicinity of the first boundary, and a second electrode positioned in the vicinity of the second boundary; wherein said electrodes are adapted to apply constant electrical potentials within said closed loop, so that electrokinetic flow within said closed loop circulates in a periodic manner as the result of the different signs of electroosmosis in said first and second branches.

4. A reactor as recited in claim 1, comprising three said temperature controllers; wherein said three temperature controllers are adapted to make the temperatures of their respective three zones about 90-95° C., about 55-65° C., and about 70-75° C., whereby said reactor is adapted to conduct multiple cycles of the polymerase chain reaction.

5. A reactor as recited in claim 4, wherein said reactor is adapted to conduct the polymerase chain reaction with an average cycle time of less than 15 seconds for a 500 base pair comparison target.

6. A reactor as recited in claim 4, wherein said reactor is adapted to conduct the polymerase chain reaction with an average cycle time of less than 10 seconds for a 500 base pair comparison target.

7. A reactor as recited in claim 4, wherein said reactor is adapted to conduct the polymerase chain reaction with an average cycle time of less than 5 seconds for a 500 base pair comparison target.

8. A reactor as recited in claim 4, wherein said reactor is adapted to conduct the polymerase chain reaction with an average cycle time of about 5 seconds For a 500 base pair comparison target.

9. A reactor as recited in claim 1, comprising two said temperature controllers; wherein said two temperature controllers are adapted to make the temperatures of their respective zones about 90-95° C., and about 60-65° C., whereby said reactor is adapted to conduct multiple cycles of the ligase detection reaction.

10. A reactor as recited in claim 1, comprising two said temperature controllers; wherein said two temperature controllers are adapted to make the temperatures of their respective zones about 90-95° C., and about 55-75° C., whereby said reactor is adapted to conduct multiple cycles of the polymerase chain reaction.

11. A reactor as recited in claim 1, additionally comprising a detector adapted to detect, online, the presence of reagents, products, or both within said closed loop.

12. A reactor as recited in claim 1, additionally comprising an optical detector adapted to detect, online, the presence of reagents, products, or both within said closed loop.

13. A reactor as recited in claim 1, additionally comprising a conductivity detector adapted to detect, online, the presence of reagents, products, or both within said closed loop.

14. A reactor as recited in claim 1, wherein air gaps or insulators separate adjacent temperature controllers, to reduce heat flow between adjacent temperature zones.

15. A reactor as recited in claim 1, wherein the volume of said closed loop reactor is between about 50 pL and about 10 mL.

16. A reactor as recited in claim 1, wherein the volume of said closed loop reactor is between about 1 mL and about 10 μL.
